(19) United States
(12) Patent Application Publication
(10) Pub. No.: US 2025/0145482 A1
(43) Pub. Date: May 8, 2025

(54) AMMONIA SYNTHESIS AND UREA SYNTHESIS WITH REDUCED CO2 FOOTPRINT

(71) Applicants: thyssenkrupp Uhde GmbH, Dortmund (DE); thyssenkrupp AG, Essen (DE)

(72) Inventors: Frederick KESSLER, Menden (DE); Yevgeny MAKHYNYA, Muelheim an der Ruhr (DE)

(73) Assignees: thyssenkrupp Uhde GmbH, Dortmund (DE); thyssenkrupp AG, Essen (DE)

(21) Appl. No.: 18/729,850

(22) PCT Filed: Jan. 19, 2023
(86) PCT No.: PCT/EP2023/051293

§ 371 Date: Jul. 17, 2024

(30) Foreign Application Priority Data
Jan. 19, 2022 (BE) BE 2022/5031
Jan. 19, 2022 (DE) 10 2022 200 573.2

(51) Int. Cl. C01C 1/04; C01B 3/02; C01B 3/36; C07C 273/10

(52) U.S. Cl. CPC C01C 1/0488; C01B 3/025; C01B 3/36; C07C 273/10; C01B 2203/0244; C01B 2203/0415; C01B 2203/0475; C01B 2203/068; C01B 2203/1235

(57) ABSTRACT

The present invention relates to an apparatus for synthesis of ammonia, in which the gases formed on the burner side of the primary reformer are used at least partly as reactants.

The invention relates to a plant for production of ammonia from a combination of hydrogen from natural gas and from electrolysis by means of renewable energies and simultaneous utilization of carbon dioxide in urea synthesis and/or of nitrogen in ammonia synthesis, which are formed in the production of hydrogen from natural gas, to an integrated plant system having a plant for synthesis of ammonia and a plant for further synthesis of urea from the ammonia produced, and to a method of increasing the capacity of an existing plant according to the prior art.

A particular method used for production of hydrogen is steam reforming, in which a hydrocarbon is reacted with steam to give carbon monoxide and hydrogen, and then the carbon monoxide is converted in a water-gas shift reaction to carbon dioxide and hydrogen. For this purpose, energy has to be provided from the outside for this endothermic reaction, which is effected, for example, by combustion of hydrocarbons in an adjacent combustion chamber. Alternatively, autothermal reforming is used, in which a partial oxidation takes place and hence provides the required energy.

Methane is typically reacted with water and air in a primary reformer and secondary reformer to give carbon dioxide and hydrogen, typically with establishment of the target composition of 3:1 of hydrogen to nitrogen. This is typically done in steps, firstly by reaction of methane with water in a primary reformer with supply of energy, and then in a secondary reformer with supply of oxygen, usually in the form of air, followed by a shift reaction for reaction of carbon monoxide with water to give carbon dioxide and hydrogen. Thus, after removal of the carbon dioxide and typically the conversion of any carbon monoxide present in methane, this mixture can be converted directly to ammonia in a converter.

After the synthesis, the ammonia is often reacted directly with carbon dioxide to give urea. For this purpose, it is often the case that the carbon dioxide from the reformer, i.e. from the process for production of the hydrogen, is used.

WO 2019/110 443 A1 discloses a method of providing CO₂ for urea synthesis from flue gas and synthesis gas.

EP 3 390 354 B1 discloses a method of providing carbon dioxide for the synthesis of urea.

It is likewise known that hydrogen can be produced by electrolysis from renewable energy sources in particular and hence in a CO₂-free manner. There is therefore of course an interest in using this “green” hydrogen for the synthesis of ammonia as well, in order to obtain “green” ammonia. Since no nitrogen is obtained here in the production of hydrogen, this is typically obtained from air fractionation, but this is an energy-intensive process.

In order to operate the process within the primary reformer, a primary reformer has a burner side on which a fuel gas, usually natural gas, is combusted with air and hence provides the necessary thermal energy. The flue gas exiting from the burner side comprises mainly nitrogen and carbon dioxide: two substances that are actually usable within the plant but are usually released directly to the environment.

It is an object of the invention to at least partly utilize the flue gas generated on the burner side of the primary reformer within the process and hence to save energy in the overall process and/or to reduce emissions.

This object is achieved by the plant having the features specified in claim 1, by the integrated plant system having the features specified in claim 13 and in claim 14, and by the methods having the features specified in claim 18 and in claim 19. Advantageous developments will be apparent from the dependent claims, the description that follows and the drawings.

The plant serves for synthesis of ammonia, and can optionally in an integrated plant system (a combined plant) together with a plant for further synthesis of urea from the ammonia produced. Such combined plants for production of nitrogen-containing fertilizer are known and customary. Such combined plants may alternatively have further or different constituents, for example a nitric acid plant for production of nitric acid from ammonia and in particular a downstream apparatus for production of ammonium nitrate as fertilizer from ammonia and nitric acid.

The plant has a reformer for conversion of a hydrocarbon to hydrogen. For example, the reformer has a primary reformer and a secondary reformer for conversion of a hydrocarbon to hydrogen; in particular, steam reforming is used here, in which, in a first step, in particular, methane is reacted with steam and in a second step with air, typically with a downstream water-gas shift reaction in which carbon monoxide produced is reacted with steam to give carbon dioxide and hydrogen. Alternatively, the reformer may be an autothermal reformer in which hydrocarbon, steam and oxygen are combined such that the energy required for the conversion to hydrogen is formed directly from the combustion. By contrast with steam reforming, there is no need to supply energy from the outside here. Moreover, the plant has a converter for conversion of hydrogen and nitrogen to ammonia. The converter has a catalyst and is operated at high pressure and high temperature. Since the conversion is an equilibrium reaction that does not have virtually complete conversion, the synthesis gas is directed into a recirculation circuit in order to be able to feed unconverted reactants back to the converter. The method is known as the Haber-Bosch process. The converter is correspondingly incorporated into a recirculation circuit. A first carbon dioxide separator is disposed between the reformer and the recirculation circuit. The carbon dioxide formed from the starting material, especially methane, is separated off here, for example and in particular in order then to supply it to a urea synthesis apparatus. Thus, downstream of the first carbon dioxide separator, a gas stream comprising nitrogen and hydrogen in a ratio of 1:3 and without further components (possibly apart from traces) is provided for the ammonia synthesis. A methanator (methanizer), an apparatus for conversion of any traces of carbon monoxide and carbon dioxide present to methane, is typically present between the first carbon dioxide separator and the recirculation circuit, in order to prevent catalyst poisoning. The recirculation circuit has an ammonia separator. The ammonia product is separated here from the unconverted reactant stream of nitrogen and hydrogen. In addition, the recirculation circuit typically has heat exchangers between the converter and the ammonia separator for cooling, and between the ammonia separator and the converter for heating. In addition, the recirculation circuit typically has a compressor.

According to the invention, the plant has a further hydrogen source. The further hydrogen source may especially be a pure hydrogen source, i.e. a hydrogen source that provides at least one gas stream having a hydrogen content of at least 90% by volume, especially of at least 95% by volume. The hydrogen from the further hydrogen source preferably does not come from a reforming process in the front end of the plant. The further hydrogen source is preferably a water electrolysis. The water electrolysis is preferably a conventional water electrolysis (for instance an acidic, alkaline or neutral water electrolysis, or a chloralkali electrolysis), a solid oxide electrolysis (for instance an SOEC electrolysis), a high-temperature electrolysis or a high-pressure electrolysis (for instance an HPE electrolysis or a UHPE electrolysis). The water electrolysis is preferably operated by means of renewable energies. The hydrogen thus produced is thus free of carbon dioxide emissions, and is thus considered to be “green” hydrogen. The further hydrogen source is connected to the recirculation circuit such that hydrogen is fed into the recirculation circuit. For this purpose, the hydrogen from the further hydrogen source is preferably combined with the hydrogen from the steam reforming, preferably downstream of the secondary reformer and further preferably downstream of a water-gas shift reaction. In this way, the hydrogen is mixed with nitrogen at low pressure, such that it is easier to compress. The plant has a combustion apparatus. The combustion apparatus is connected to the reformer. In this case, the combustion apparatus may also be part of the reformer, as shown in embodiments hereinafter. For example, the combustion apparatus may be the burner side of a primary reformer. Alternatively, the combustion apparatus may be a steam generator. A steam generator is operated, for example, for operation of the compressors. It is likewise also possible to combine the offgases from two or more combustion apparatuses if a larger gas stream is desired. The combustion apparatus, for example the burner side of the primary reformer, is connected to the secondary reformer. In this way, nitrogen, but also carbon dioxide and residual oxygen, are fed into the gas stream for generation of hydrogen. The remaining residual oxygen is converted in the secondary reformer. Since carbon dioxide is separated off downstream of the secondary reformer, it is thus also possible to separate off the carbon dioxide generated on the burner side in the same step.

In a further embodiment of the invention, the further hydrogen source is connected to the recirculation circuit such that hydrogen is fed into the recirculation circuit, for which the hydrogen is preferably first mixed with nitrogen and then compressed by means of one or more compressors. The plant has a combustion apparatus. For example, the combustion apparatus may be the burner side of a primary reformer. Alternatively, the combustion apparatus may be a steam generator. A steam generator is operated, for example, for operation of the compressors. It is likewise also possible to combine the offgases from two or more combustion apparatuses if a larger gas stream is desired. The combustion apparatus, for example the burner side of the primary reformer, is connected to a second carbon dioxide separator. The second carbon dioxide separator is connected to the recirculation circuit such that nitrogen is fed into the recirculation circuit. Preferably, the hydrogen from the further hydrogen source and the nitrogen from the second carbon dioxide separator are first combined and compressed collectively by means of one or more compressors. The advantage is that the combustion on the burner side can result in comparatively simple provision of nitrogen in further process steps by a separation from the carbon dioxide, and hence establishment of the ratio of hydrogen to nitrogen of 3:1 without energy-intensive air fractionation.

In a further embodiment of the invention, the reformer has a primary reformer and a secondary reformer for conversion of a hydrocarbon to hydrogen. The primary reformer has a hydrogen side and a burner side. On the hydrogen side, hydrocarbon is reacted with steam to give carbon monoxide or carbon dioxide and hydrogen. The energy needed for the purpose is provided by combustion, especially of hydrocarbon with oxygen, especially with air. The burner side here is the combustion apparatus; hydrocarbon is combusted with air on the burner side of the primary reformer. The burner side of the primary reformer is connected to a second carbon dioxide separator.

In a further embodiment of the invention, the combustion apparatus is a steam generator.

In a further embodiment of the invention, the reformer is an autothermal reformer.

In a further embodiment of the invention, the second carbon dioxide separator is an ammonia-water scrubber. Such scrubbers are known, for example, from WO 2019/110 443 A1 or EP 3 390 354 B1.

In a further embodiment of the invention, the nitrogen from the second carbon dioxide separator is fed into the recirculation circuit by introducing the nitrogen from the second carbon dioxide separator into the secondary reformer. As a result, residual oxygen still present is converted in the secondary reformer. In this case, there is no need to transfer the complete nitrogen stream into the secondary reformer. Instead, this stream can be matched to the amount of additional hydrogen.

In a further embodiment of the invention, an apparatus for removal of oxygen is disposed between the second carbon dioxide separator and the recirculation circuit. In that case, an additional compressor is preferably provided in order to achieve matching of the pressure to the high level of the recirculation circuit.

In a further embodiment of the invention, the burner side of the primary reformer is operated with a deficiency of oxygen or an excess of methane. This is unusual for the actual operation as burner side, but this ensures that the oxygen is fully consumed.

In a further embodiment of the invention, the second carbon dioxide separator is connected to the recirculation circuit such that nitrogen is fed into the recirculation circuit via the secondary reformer. In a further embodiment of the invention, the second carbon dioxide separator is connected to the recirculation circuit such that nitrogen is fed into the recirculation circuit via the autothermal reformer. In this way too, the existing residual oxygen can be reliably converted.

In a further embodiment of the invention, a dedusting apparatus is disposed between the combustion apparatus, preferably the burner side of the primary reformer, and the second carbon dioxide separator. There may preferably additionally be a desulfurizing apparatus and/or a denoxing apparatus downstream of the dedusting apparatus.

In a further embodiment of the invention, the further hydrogen source and the second carbon dioxide separator are connected to the recirculation circuit such that the hydrogen stream from the further hydrogen source is first combined with the nitrogen stream from the second carbon dioxide separator, then is guided through a first compressor and thereafter is guided through a methanator and then fed into the recirculation circuit. This especially enables a simplified increase in capacity of the ammonia synthesis, since the existing synthesis gas production in the reformer remains unchanged and hence is increased by the additional gas stream directly upstream of the converter.

In a further embodiment of the invention, a dedusting apparatus is disposed between the burner side of the primary reformer and the secondary reformer. There may preferably additionally be a desulfurizing apparatus and/or a denoxing apparatus downstream of the dedusting apparatus.

In a further embodiment of the invention, a compressor is disposed between the combustion apparatus, preferably the burner side of the primary reformer, and the reformer, preferably the secondary reformer.

In a further aspect, the invention relates to an integrated plant system having the above-described plant for synthesis of ammonia and a plant for further synthesis of urea from the ammonia produced, wherein the integrated plant system also has a urea synthesis apparatus for synthesis of urea from ammonia and carbon dioxide. The first carbon dioxide separator for the carbon dioxide separated is connected to the urea synthesis apparatus. Typically, the amount of carbon dioxide separated is somewhat smaller than the amount of ammonia produced from the nitrogen and hydrogen, such that conversion of the ammonia to urea is incomplete. The ammonia separator has an ammonia-conducting connection to the urea synthesis apparatus. In this case, there may also be an intermediate reservoir disposed in the ammonia-conducting connection.

In a further embodiment of the invention, the integrated plant system likewise serves for further synthesis of urea from the ammonia produced. Such combined plants for production of nitrogen fertilizers are known and customary. These combined plants may also have further or different constituents, for example a nitric acid plant for production of nitric acid from ammonia, and in particular a downstream apparatus for production of ammonium nitrate as fertilizer from ammonia and nitric acid. The integrated plant system also has a urea synthesis apparatus for synthesis of urea from ammonia and carbon dioxide. A first carbon dioxide separator is disposed between the reformer and the recirculation circuit. Here, the carbon dioxide formed from the starting material, especially from methane, is separated off, for example and in particular in order then to feed it to the urea synthesis apparatus. What is thus provided downstream of the first carbon dioxide separator is a gas stream comprising nitrogen and hydrogen in a ratio of 1:3 and without further components (possibly apart from traces) for the ammonia synthesis. The ammonia separator has an ammonia-conducting connection to the urea synthesis apparatus. In this case, there may also be an intermediate reservoir disposed in the ammonia-conducting conduit. The combustion apparatus, for example the burner side of the primary reformer, is connected to a second carbon dioxide separator. The second carbon dioxide separator is connected to the urea synthesis apparatus such that carbon dioxide is fed into the urea synthesis apparatus.

In a further embodiment of the invention, the first carbon dioxide separator for the carbon dioxide separated is connected to the urea synthesis apparatus. Typically, the amount of carbon dioxide separated is somewhat smaller than the amount of ammonia produced from the nitrogen and hydrogen, and so conversion of the ammonia to urea is incomplete.

In a further embodiment of the invention, the second carbon dioxide separator is an ammonia-water scrubber. Such scrubbers are known, for example, from WO 2019/110 443 A1 or EP 3 390 354 B1.

In a further embodiment of the invention, a dedusting apparatus is disposed between the combustion apparatus, preferably the burner side of the primary reformer, and the second carbon dioxide separator.

In a further embodiment of the invention, in the integrated plant system, the burner side of the primary reformer is connected to a second carbon dioxide separator. The second carbon dioxide separator is connected to the recirculation circuit such that nitrogen is fed into the recirculation circuit. The burner side of the primary reformer is connected to the secondary reformer.

In a further embodiment of the invention, in the integrated plant system, the burner side of the primary reformer is likewise connected to a second carbon dioxide separator. The second carbon dioxide separator is connected to the recirculation circuit such that nitrogen is fed into the recirculation circuit. Moreover, the second carbon dioxide separator is connected to the urea synthesis apparatus such that carbon dioxide is fed into the urea synthesis apparatus. This can achieve optimal utilization of all gas streams. By virtue of the hydrogen additionally produced, especially “green” hydrogen, it is possible to achieve a ratio of nitrogen to hydrogen to carbon dioxide of, for example, 2:6:1.

In a further embodiment of the invention, in the integrated plant system, the burner side of the primary reformer is connected to the secondary reformer. The burner side of the primary reformer is also connected to a second carbon dioxide separator, and the second carbon dioxide separator is connected to the urea synthesis apparatus such that carbon dioxide is fed into the urea synthesis apparatus.

In this case, not all gas streams must always be guided entirely in accordance with the interconnection. For example, in the different embodiments, it is also possible to separate off substreams, especially the offgas from the burner side, the nitrogen stream or the carbon dioxide stream from the second carbon dioxide separator and discard them or use them in some other way.

In a further aspect, the invention relates to a method of increasing the capacity of an existing plant according to the prior art. In this case, the plant is extended to include a further hydrogen source, especially a pure hydrogen source, especially a water electrolysis. The water electrolysis is preferably operated by means of renewable energies. The hydrogen thus produced is thus free of carbon dioxide emissions, and is thus considered to be “green” hydrogen. The further hydrogen source is connected here to the recirculation circuit such that hydrogen is fed into the recirculation circuit. This can increase the capacity of the ammonia synthesis. The burner side of the primary reformer is connected to the secondary reformer. As a result, the synthesis is supplied firstly with nitrogen and secondly with carbon dioxide. The carbon dioxide is separated off together with the carbon dioxide formed in the reformer and fed into the urea synthesis. In this way, it is firstly possible to increase the overall capacity; secondly, the carbon dioxide footprint is reduced.

In a further aspect, the invention relates to a further method of increasing the capacity of an existing integrated plant system according to the prior art having a plant for synthesis of ammonia and a plant for further synthesis of urea from the ammonia produced. The integrated plant system is extended to include a further hydrogen source and a second carbon dioxide separator, especially a pure hydrogen source, especially a water electrolysis. The water electrolysis is preferably operated by means of renewable energies. The hydrogen thus produced is thus free of carbon dioxide emissions, and is thus considered to be “green” hydrogen. The further hydrogen source is connected to the recirculation circuit such that hydrogen is fed into the recirculation circuit. This increases the amount of hydrogen fed to the converter. The burner side of the primary reformer is connected to the second carbon dioxide separator. Moreover, the second carbon dioxide separator is connected to the recirculation circuit such that nitrogen is fed into the recirculation circuit. Thus, as well as additional hydrogen, nitrogen is also fed in, and hence the total capacity is increased. Moreover, the second carbon dioxide separator is connected to the urea synthesis apparatus such that carbon dioxide is fed into the urea synthesis apparatus. This ensures increased production of urea by virtue of the elevated amount of ammonia.

The plant of the invention and the integrated plant system of the invention are elucidated in detail hereinafter by working examples shown in the drawings.

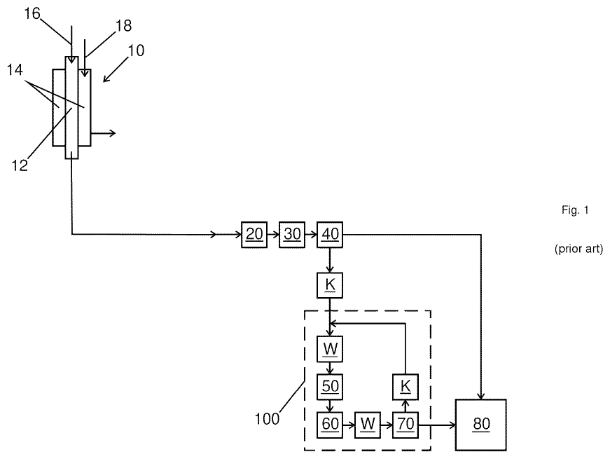

FIG. 1 prior art

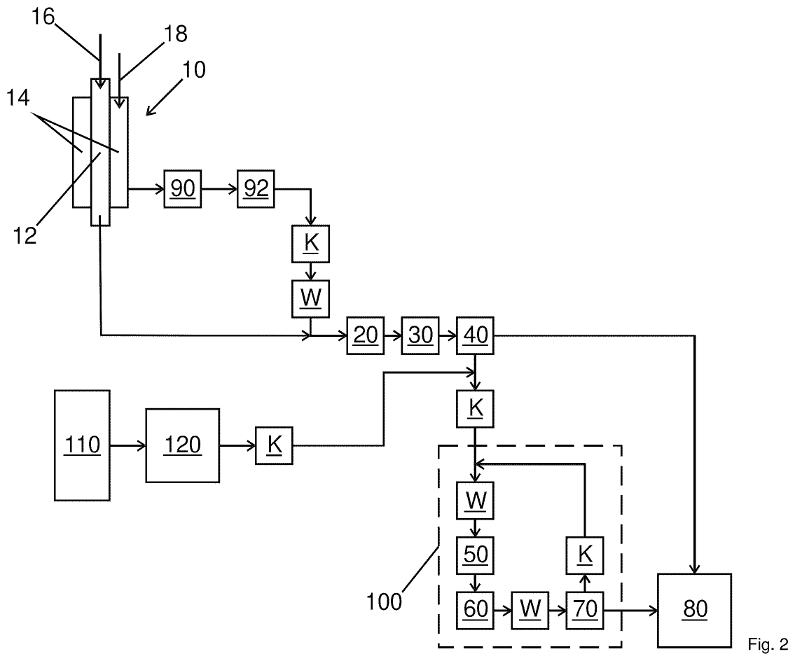

FIG. 2 third illustrative embodiment

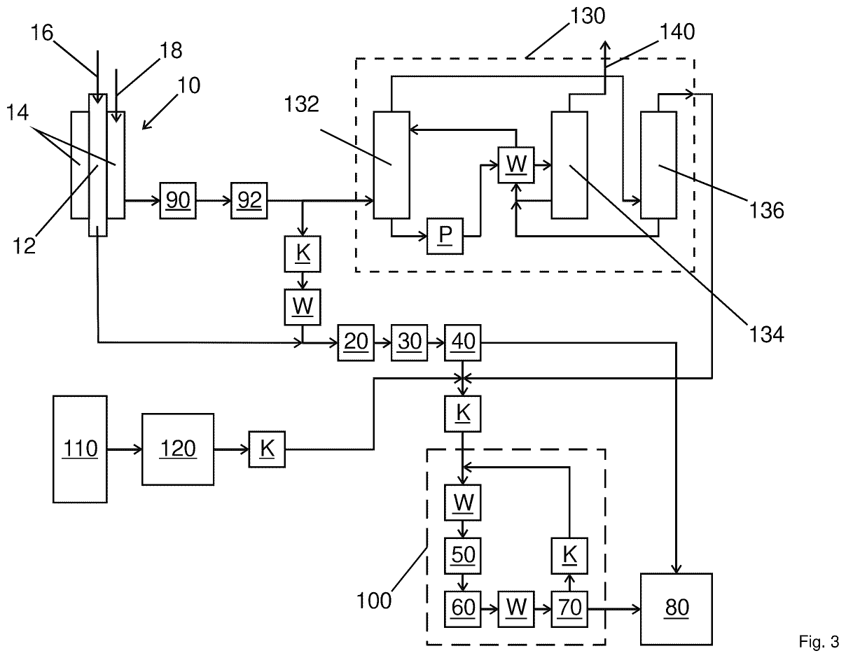

FIG. 3 fifth illustrative embodiment

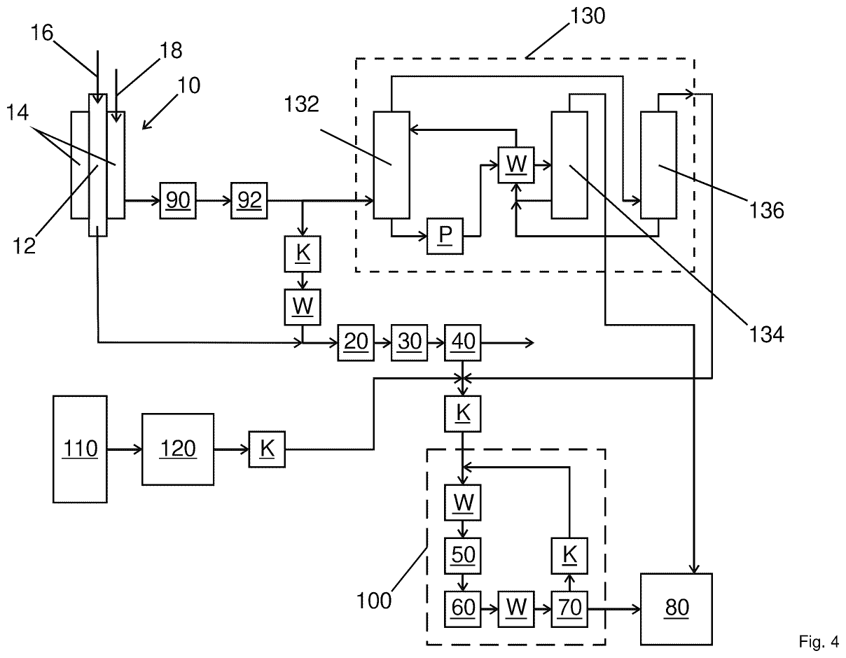

FIG. 4 sixth illustrative embodiment

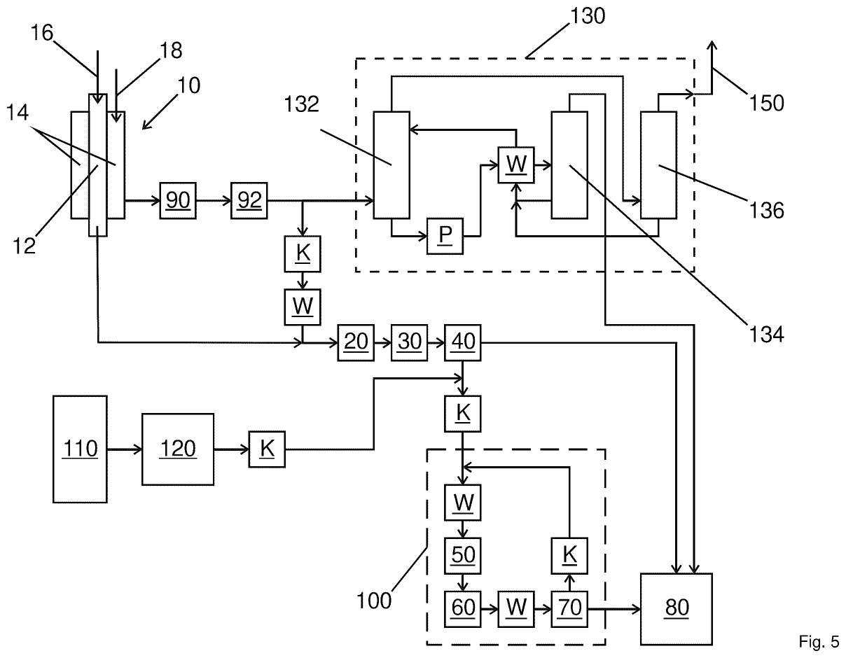

FIG. 5 eighth illustrative embodiment

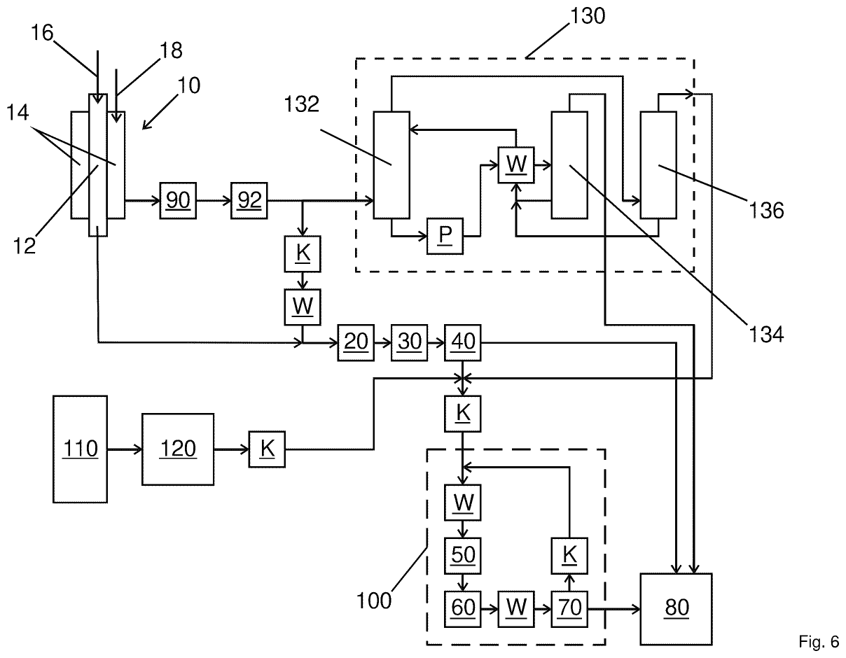

FIG. 6 ninth illustrative embodiment

First of all, the constituents that are common to all working examples will be addressed, shown by the prior art in FIG. 1, and then subsequently only the additional components in each case.

The diagrams are simplified and merely schematic. For example, compressors K may also be in multistage form. Moreover, what is called a methanator is typically present, which is disposed upstream of the feed to the recirculation circuit 100 and converts residual amounts of carbon dioxide and carbon monoxide, which are catalyst poisons, to methane. Such variants that are customary for ammonia synthesis are not shown here by way of simplification. It is likewise possible for the two compressors disposed downstream of the first carbon dioxide separator 40 and the ammonia separator 70 to be identical. Such variants and arrangements for conduction of gas are known to the person skilled in the art and have no direct effect on the invention.

The integrated plant system according to the prior art as per FIG. 1 serves for synthesis of ammonia with further conversion to urea, wherein the hydrogen is produced by steam reforming, and ammonia via the Haber-Bosch process.

In a primary reformer 10, methane and steam are supplied on the hydrogen side 12 as hydrogen source 16. The energy required for the conversion is generated and provided by combustion on the burner side 14. For example, a mixture of methane and air is provided via the fuel gas feed 18. What is thus ideally produced on the burner side 14 is a gas mixture of nitrogen and carbon dioxide. In reality, there may be about 2% by volume of oxygen present as a further component. The gas mixture generated on the hydrogen side 12 is conducted into a secondary reformer 20, where air is typically added. For example, methane is reacted here with oxygen to give carbon monoxide and hydrogen. In a downstream shift reactor 30, typically consisting of two separate reactors at different temperatures, carbon monoxide is reacted with water to give carbon dioxide and hydrogen. Subsequently, in a first carbon dioxide separator 40, the carbon dioxide is separated off. The gas, which should then only contain nitrogen and hydrogen, is guided via a compressor K into the recirculation circuit 100. In the recirculation circuit 100, the gas is first heated in a heat exchanger W and then fed into the converter 50. Subsequently, in a cooler 60, the heat of reaction released in the conversion is removed. Subsequently, the gas stream is cooled down further in a heat exchanger W, such that ammonia is separated off in the ammonia separator 70. Unconverted hydrogen and unconverted nitrogen remain in the gas stream. These gases are recycled by means of a compressor, so as to give rise to the recirculation circuit 100. The ammonia separated out in the ammonia separator 70 and the carbon dioxide separated off in the first carbon dioxide separator are converted to urea and water in the urea synthesis apparatus 80. This is typically followed by a pelletization, with or without further additives, in order to sell the urea as fertilizer.

The illustrative embodiments will now be shown hereinafter with reference to the additional components and connections.

FIG. 2 shows a third illustrative embodiment. This includes a further hydrogen source. The latter consists merely by way of example of a solar and wind park 110. Power is generated here from the renewable energies of sun and wind. This power is utilized for generation of hydrogen in the water electrolysis 120. The hydrogen can be stored intermediately in a reservoir in order to compensate for fluctuations in insolation and wind. Accordingly, it is likewise possible for there to be a battery for balancing between the solar and wind park 110 and the water electrolysis 120. The (“green”) hydrogen thus generated is combined with the gas stream coming from the reformer and fed into the recirculation circuit 100. As a result, however, nitrogen is present in a substoichiometric amount. In order not to have to implement an energy-intensive air fractionation, the nitrogen is obtained from the offgas from the burner side 14 of the primary reformer 10. For this purpose, the gas is first dedusted in a dedusting apparatus 90. Optionally, the gas can then be conducted through a desulfurizing apparatus 92, especially in regions in which sulfur-containing natural gas is used. Subsequently, the gas is fed via a compressor K and a heat exchanger W into the secondary reformer 20. It is also possible here for the sequence of compressor K and heat exchanger W to be reversed. As a result, firstly, the ratio of hydrogen to nitrogen is balanced out again. Secondly, more carbon dioxide is introduced, which is separated out in the first carbon dioxide separator 40 and fed to the urea synthesis apparatus 80. As a result, in a very simple manner, it is possible to increase the total amount of urea produced and at the same time to reduce the CO2 footprint.

FIG. 3 showed a fifth illustrative embodiment. In this embodiment, there is likewise a further hydrogen source. This consists merely by way of example of a solar and wind park 110. Power is generated here from the renewable energies of sun and wind. This power is utilized for generation of hydrogen in the water electrolysis 120. The hydrogen can be stored intermediately in a reservoir in order to compensate for fluctuations in insolation and wind. Accordingly, it is likewise possible for there to be a battery for balancing between the solar and wind park 110 and the water electrolysis 120. The (“green”) hydrogen thus generated is combined with the gas stream coming from the reformer and fed into the recirculation circuit 100. As a result, however, nitrogen is present in a substoichiometric amount. In order not to have to implement an energy-intensive air fractionation, the nitrogen is obtained from the offgas from the burner side 14 of the primary reformer 10. For this purpose, the gas is first dedusted in a dedusting apparatus 90. Optionally, the gas can then be conducted through a desulfurizing apparatus 92, especially in regions in which sulfur-containing natural gas is used. Subsequently, the gas is conducted into the second carbon dioxide separator 130, in the form of an ammonia-water scrubber, as can be inferred for example from WO 2019/110 443 A1 or EP 3 390 354 B1. The second carbon dioxide separator 130 has a CO₂ dissolution apparatus 132 in which the carbon dioxide is dissolved in aqueous ammonia. The solution is then compressed to 150 bar, for example, by means of a pump P and guided via a heat exchanger W into the CO₂ release apparatus 134. The carbon dioxide is released again therein at elevated temperatures and can be released via the CO₂ outlet 140. In the simplest case, it is released to the environment. It can alternatively be stored or converted in order to avoid CO₂ emissions. The aqueous ammonia is conducted from the CO₂ release apparatus 134 via the heat exchanger W back into the CO₂ dissolution apparatus 132. In addition, the second carbon dioxide separator 130 has an ammonia retention scrub 136. This affords a pure nitrogen stream, which is then fed into the recirculation circuit 100 supplied gas stream. In this case, it is also possible to feed in only part of the nitrogen gas stream, in order to obtain the correct stoichiometry. Excess nitrogen can, for example, simply be released to the environment or be used as inert gas in further syntheses. Since oxygen and nitrogen are similar than nitrogen and carbon dioxide, separation from this gas stream from the burner side 14 is more efficient than air fractionation. The advantage of this fifth illustrative embodiment is the flexible conduction in order to feed in a portion of the carbon dioxide from the burner side 14 of the urea synthesis apparatus 80 and to release another portion via the CO₂ outlet 140, in order thus to be able to easily establish the correct stoichiometry. It is also possible here for the sequence of compressor K and heat exchanger to be reversed.

FIG. 4 showed a sixth illustrative embodiment, which differs from the fifth embodiment in that the carbon dioxide from the second carbon dioxide separator 130 is used in the urea synthesis apparatus 80. For this purpose, the carbon dioxide obtained in the first carbon dioxide separator 40 is discarded, since it is at a lower pressure level.

FIG. 5 showed an eighth illustrative embodiment. In many combined plants, less carbon dioxide is provided from the first carbon dioxide separator 40 than would be needed for complete conversion of the ammonia to urea. In order to increase production, there is thus a need to find a further carbon dioxide source. This exists in the offgas from the burner side 14 of the primary reformer 10. For this purpose, the gas is first dedusted in a dedusting apparatus 90. Optionally, the gas can then be guided through a desulfurizing apparatus 92, especially in regions in which sulfur-containing natural gas is used. Subsequently, the gas is conducted into the second carbon dioxide separator 130, in the form of an ammonia-water scrubber, as can be inferred, for example, from WO 2019/110 443 A1 or EP 3 390 354 B1. The second carbon dioxide separator 130 has a CO₂ dissolution apparatus 132, in which the carbon dioxide is dissolved in aqueous ammonia. The solution is then compressed to 150 bar, for example, by means of a pump P and guided via a heat exchanger W into the CO₂ release apparatus 134. The carbon dioxide is released again therein at elevated temperatures is then fed into the urea synthesis apparatus 80, where the high pressure of the CO₂ release apparatus 134 provides the carbon dioxide at the correct pressure level. The aqueous ammonia is conducted from the CO₂ release apparatus 134 via the heat exchanger W back into the CO₂ dissolution apparatus 132. In addition, the second carbon dioxide separator 130 has an ammonia retention scrub 136, as a result of which no ammonia is released into the environment with the nitrogen via the nitrogen outlet 150 or is introduced into further syntheses with the nitrogen as inert gas. In this case, the nitrogen required for the renewably generated hydrogen is provided via the gas stream fed into the secondary reformer 20. At the same time, further carbon dioxide is provided via the second carbon dioxide separator 130 of the urea synthesis apparatus 80.

FIG. 6 showed a ninth illustrative embodiment. By virtue of this, all options are open during the operation of the plant in order to enable various modes of operation, for example in order to be able to adapt to fluctuating amounts of renewable energy.

### REFERENCE NUMERALS

10 primary reformer
12 hydrogen side
14 burner side
16 hydrogen source
18 fuel gas feed
20 secondary reformer
30 shift reactor
40 first carbon dioxide separator
50 converter
60 cooler
70 ammonia separator
80 urea synthesis apparatus
90 dedusting apparatus
92 desulfurizing apparatus
100 recirculation circuit
110 solar and wind park
120 water electrolysis
130 second carbon dioxide separator
132 CO₂ dissolution apparatus
134 CO₂ release apparatus
136 ammonia retention scrub
140 CO₂ outlet
150 nitrogen outlet
K compressor
P pump
W heat exchanger

1-19. (canceled)

20. A plant for synthesis of ammonia, comprising:
a reformer for conversion of a hydrocarbon to hydrogen;
a converter for conversion of hydrogen and nitrogen to ammonia, wherein the converter is incorporated into a recirculation circuit, wherein a first carbon dioxide separator is disposed between the reformer and the recirculation circuit, wherein the recirculation circuit has an ammonia separator;
a further hydrogen source connected to the recirculation circuit such that hydrogen is fed into the recirculation circuit; and
a combustion apparatus connected to the reformer.

21. The plant as claimed in claim 20, wherein the reformer has a primary reformer and a secondary reformer for conversion of a hydrocarbon to hydrogen, wherein the primary reformer has a hydrogen side and a burner side, wherein the burner side is the combustion apparatus, wherein hydrocarbon is combusted with air on the burner side of the primary reformer, wherein the burner side of the primary reformer is connected to a second carbon dioxide separator.

22. The plant as claimed in claim 21, wherein the second carbon dioxide separator is connected to the recirculation circuit such that nitrogen is fed into the recirculation circuit via the secondary reformer.

23. The plant as claimed in claim 21, wherein the reformer is an autothermal reformer.

24. The plant as claimed in claim 23, wherein the second carbon dioxide separator is connected to the recirculation circuit such that nitrogen is fed into the recirculation circuit via the autothermal reformer.

25. The plant as claimed in claim 20, wherein the combustion apparatus is a steam generator.

26. The plant as claimed in claim 20, wherein a dedusting apparatus is disposed between the combustion apparatus and the reformer.

27. The plant as claimed in claim 20, wherein a compressor is disposed between the combustion apparatus and the reformer.

28. The plant as claimed in claim 20, wherein the combustion apparatus is connected to a second carbon dioxide separator, wherein the second carbon dioxide separator is connected to the recirculation circuit such that nitrogen is fed into the recirculation circuit.

29. The plant as claimed in claim 28, wherein the second carbon dioxide separator is an ammonia-water scrubber.

30. The plant as claimed in claim 28, wherein a dedusting apparatus is disposed between the combustion apparatus and the second carbon dioxide separator.

31. The plant as claimed in claim 28, wherein the further hydrogen source and the second carbon dioxide separator are connected to the recirculation circuit such that the hydrogen stream from the further hydrogen source is first combined with the nitrogen stream from the second carbon dioxide separator, then is guided through a first compressor and thereafter is guided through a methanator and then fed into the recirculation circuit.

32. An integrated plant system comprising:
a plant for synthesis of ammonia as claimed in claim 20; and
a plant for further synthesis of urea from the ammonia produced, including a urea synthesis apparatus for synthesis of urea from ammonia and carbon dioxide;
wherein the first carbon dioxide separator for the carbon dioxide separated is connected to the urea synthesis apparatus;
wherein the ammonia separator has an ammonia-conducting connection to the urea synthesis apparatus.

33. The integrated plant system as claimed in claim 32, further comprising a combustion apparatus connected to a second carbon dioxide separator, wherein the second carbon dioxide separator is connected to the urea synthesis apparatus such that carbon dioxide is fed into the urea synthesis apparatus.

34. The integrated plant system as claimed in claim 33, wherein the second carbon dioxide separator is an ammonia-water scrubber.

35. The integrated plant system as claimed in claim 33, wherein a dedusting apparatus is disposed between the combustion apparatus and the second carbon dioxide separator.

36. A method of increasing a capacity of an existing plant for synthesis of ammonia, comprising:
extending the plant to include a further hydrogen source, wherein the further hydrogen source is connected to a recirculation circuit such that hydrogen is fed into the recirculation circuit, wherein a burner side of a primary reformer is connected to a secondary reformer.

37. A method of increasing a capacity of an existing integrated plant system including a plant for synthesis of ammonia and a plant for further synthesis of urea from the ammonia produced, the method comprising:
extending the integrated plant system to include a further hydrogen source and a second carbon dioxide separator, wherein the further hydrogen source is connected to a recirculation circuit such that hydrogen is fed into the recirculation circuit, wherein a burner side of a primary reformer is connected to the second carbon dioxide separator, wherein the second carbon dioxide separator is connected to the recirculation circuit such that nitrogen is fed into the recirculation circuit, wherein the second carbon dioxide separator is connected to a urea synthesis apparatus such that carbon dioxide is fed into the urea synthesis apparatus.
